(19) United States
(12) Patent Application Publication
(10) Pub. No.: US 2011/0059193 A1
(43) Pub. Date: Mar. 10, 2011

(54) METHOD FOR OBTAINING AN EXTRACT OF CRANBERRY MARC THAT CAN BE USED IN PARTICULAR IN THE PREVENTION AND TREATMENT OF CONDITIONS SUCH AS CARIES, GINGIVITIS OR SORE THROATS

(75) Inventors: David Tournay, Paris (FR); MIchael Tournay, Sainte Foy La Grande (FR)

(73) Assignee: TOURNAY BIOTECHNOLOGIES, SAS, Lamothe Montravel (FR)

(21) Appl. No.: 12/990,975

(22) PCT Filed: May 5, 2009
(86) PCT No.: PCT/FR09/00523

§ 371 Date: Nov. 4, 2010

(30) Foreign Application Priority Data
May 5, 2008 (FR) 0802463

(51) Int. Cl. A61K 36/45; A61K 8/97; A61P 1/02

(52) U.S. Cl. 424/732; 424/58

(57) ABSTRACT

A a method for obtaining an extract prepared from cranberry (genus Vaccinium macrocarpon and/or Vaccinium oxycoccus) marc, with a view to obtaining a product that, provided in various galenical forms or other, has, owing to the high concentration of proanthocyanidins, in particular type A proanthocyanidins, therein, many therapeutic qualities and properties that can be exploited in particular in the field of dental care and prevention and oral hygiene, and also in the field of the prevention of viral or bacterial infections of the oropharynx in humans and animals. It also relates to this extract and to uses thereof.

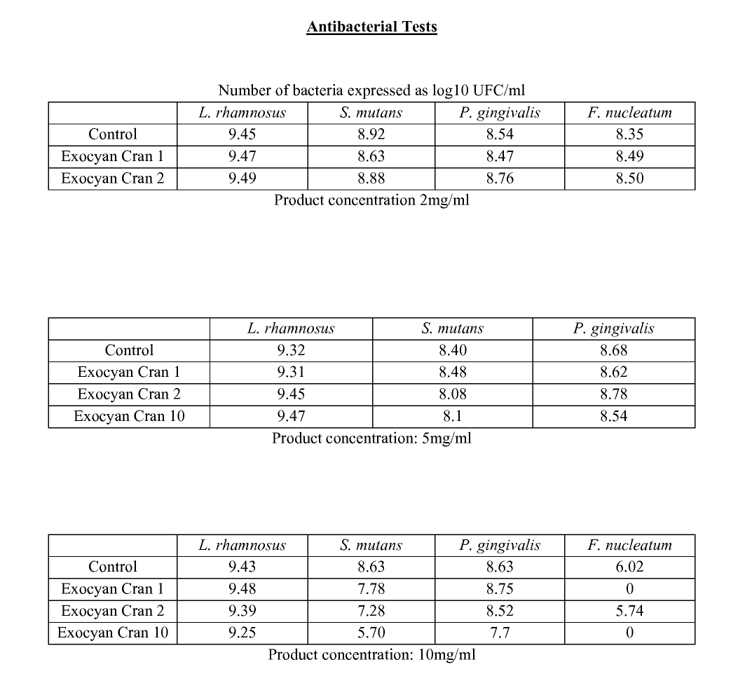

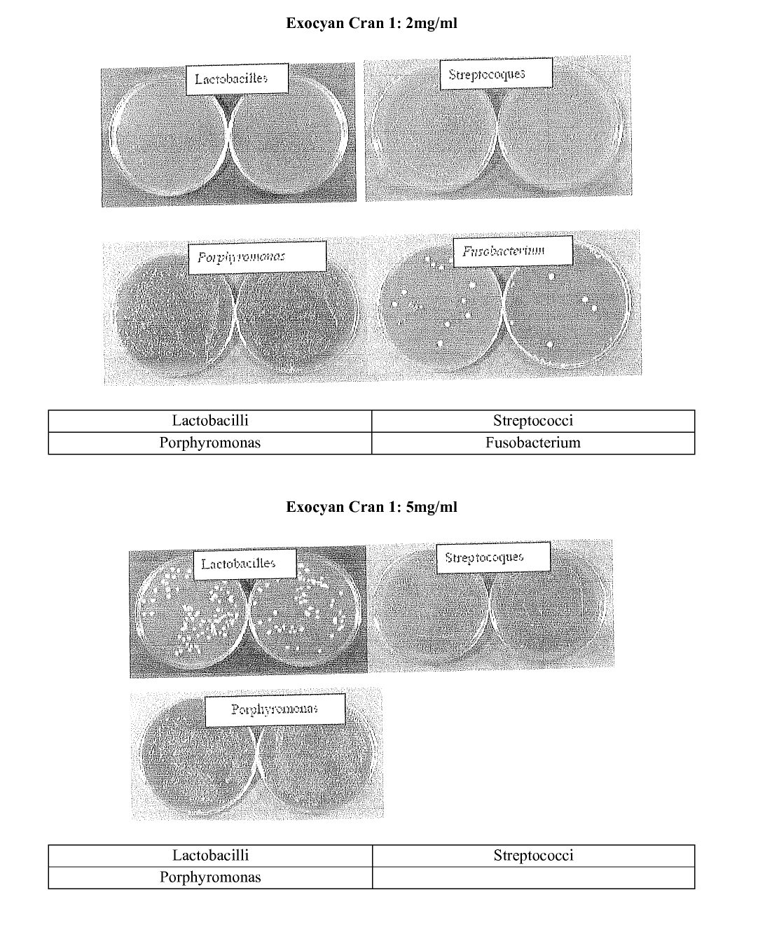

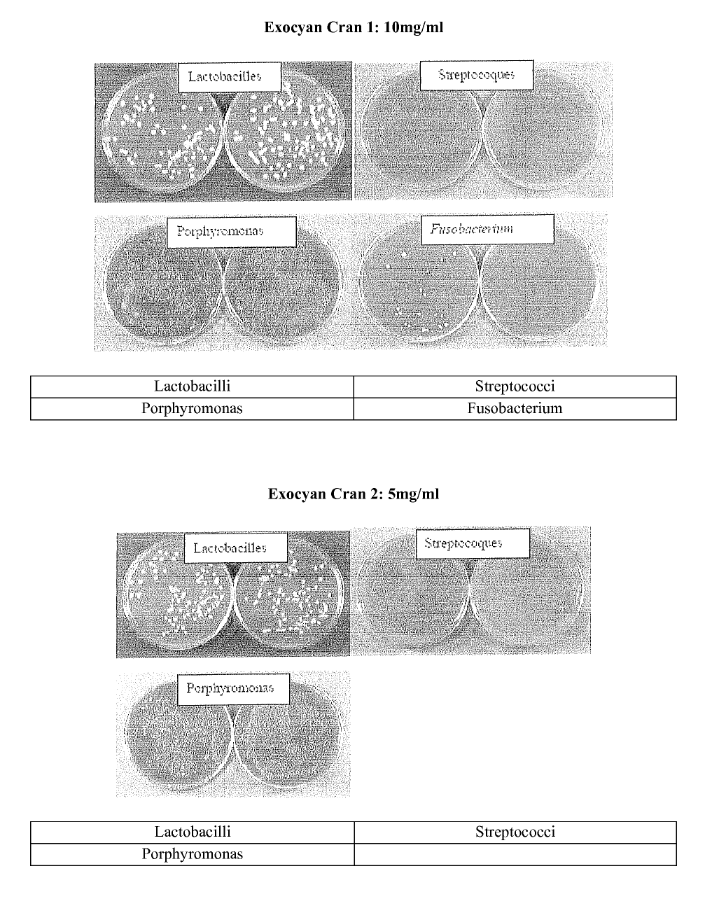

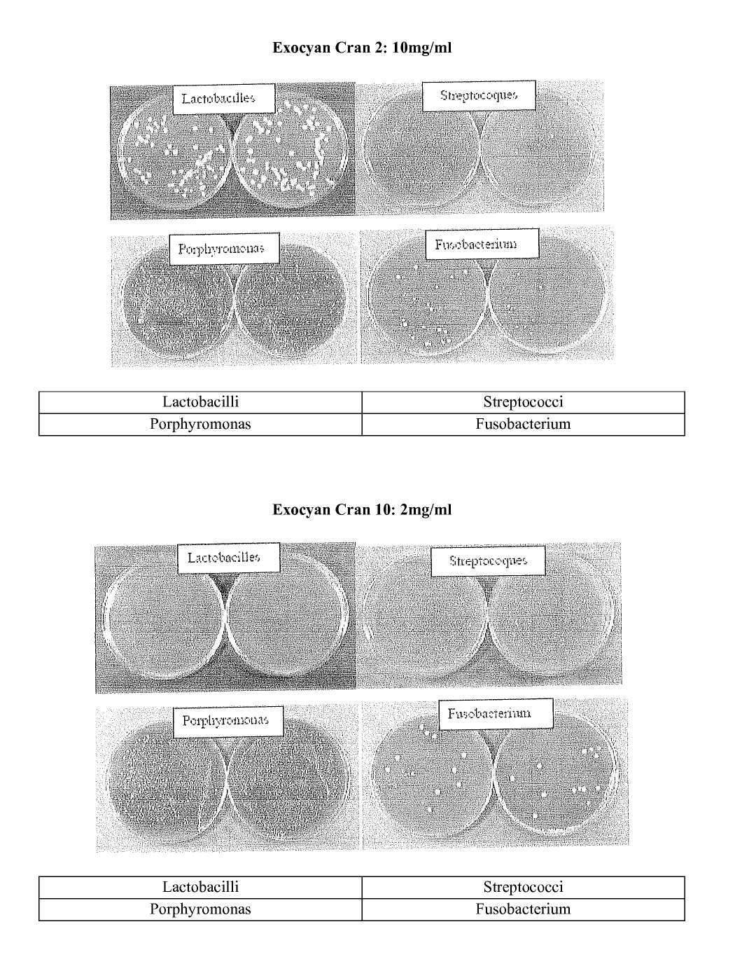

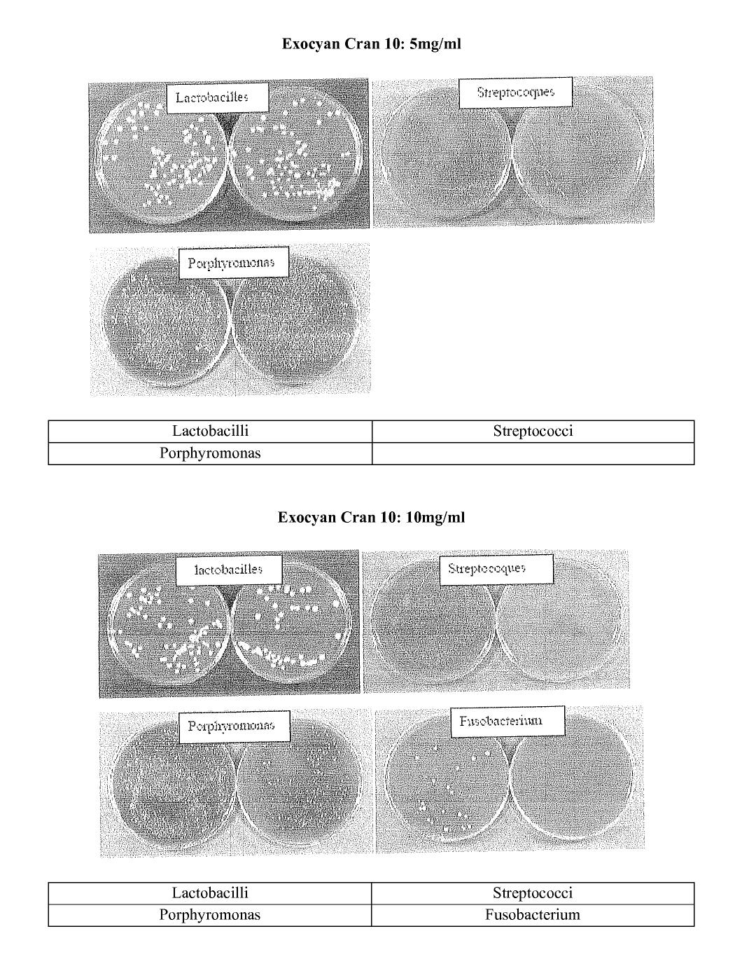

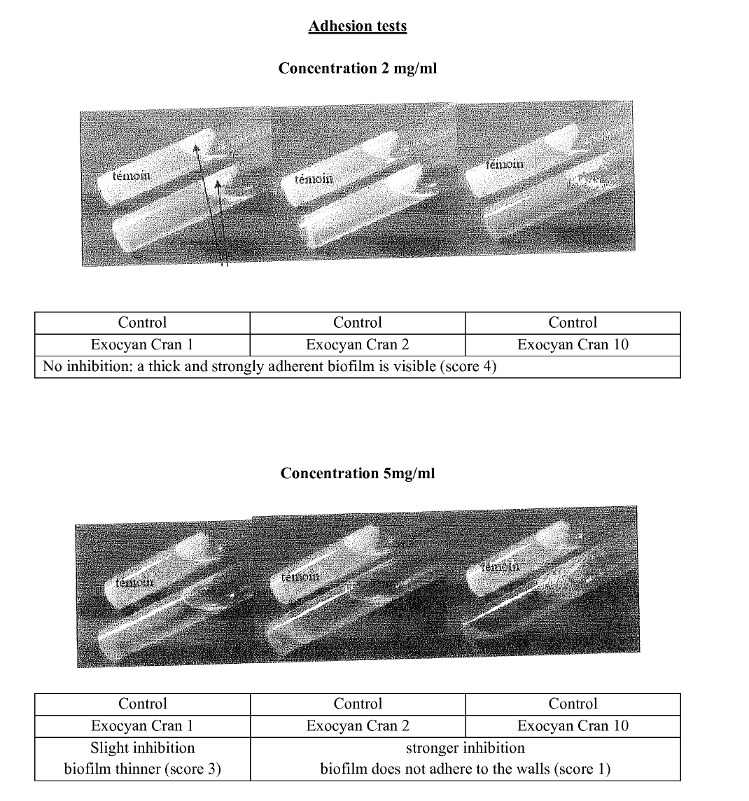

The present invention relates to a method for obtaining an extract prepared from cranberry marc (genus Vaccinium macrocarpon and/or Vaccinium oxycoccus), with a view to obtaining a product that, provided in various galenic or other forms, has many therapeutic qualities and properties that can be exploited in particular in the field of dental care and prevention and oral hygiene, and also in the field of the prevention of viral or bacterial infections of the oropharynx. It also relates to this extract and to uses thereof.

The therapeutic qualities of cranberries have been known for a long time. Mistakenly assigned at first to their high acidity due to the presence of organic acids in the juice, they have mainly been used to prevent and treat light urinary tract infections. It is now known that its therapeutic qualities are instead due to polyphenols in the juice and skin of the berry, and more specifically, proanthocyanidins, especially type A (Howell et al. 2005) which, according to AFSSA [the French Food Safety Agency], have the property of “helping reduce the adhesion (therefore they have a anti-adhesive effect) of some pathogenic bacteria of the E. coli type, on the walls of the urinary tract”.

This type of preparation has found few applications to date because of the disadvantages it can have in some cases; thus, cranberry juice, which is both bitter and sour at the same time (pH 2-3) and that must be heavily sweetened, cannot be used as-is for dental prevention or care because it can contribute to enamel erosion and the development of caries.

According to the present invention, the method for obtaining the basic product that could be usable in many applications does not exist in the prior art.

U.S. Pat. No. 5,002,759 and U.S. Pat. No. 5,362,480 relate to preparations that oppose the adhesion of pathogenic bacteria to the cells of the buccal wall; neither the genus Vaccinium nor cranberries are mentioned to describe the extract.

U.S. Pat. No. 5,474,774 relates to a preparation made from cranberries, rich in polyphenols, without simple sugars and having an anti-adhesive effect, but the extraction method and the raw material obtained are very different from the present application.

Patent US 0048611 A1 describes a cranberry-based preparation used for manufacturing a chewing gum stick having an anti-adhesive action on bacteria. In this case, the raw material used is a concentrated cranberry juice, not the marc, which is subjected to purification by dialysis. In the present application, the raw material is a completely dehydrated extract of cranberry marc and the dialysis technique is not used in its preparation.

U.S. Pat. No. 4,857,327 A1 uses a Vaccinium-based preparation to treat digestive disorders. The species Vaccinium macrocarpon and Vaccinium oxycoccus are not used: only Vaccinium myrtillus, Vaccinium vitis idea, Sambuccus nigra and Vitis vinefera are cited, none of which have proanthocyanidins type A, only those of type B. The cranberry marc used, without juice, is desiccated and included as-is into the preparation; in the present application, an extract prepared from this marc is concerned.

Patent W099/12541 relates to a cranberry extract rich in proanthocyanidins, prepared from the fruit, leaves, roots or stems of the plant. The physiological mode of action claimed mentions only the inhibition of adhesion of the E. coli bacteria onto cells of the digestive and urinary tracts, while the present application relates to action not specific to E. coli. The bacteria involved are of the Streptococcus mutans, Porphyromonas gingivalis and Fusobacterium nucleatum that are responsible, inter alia, for caries, gingivitis, periodontitis; some viruses are also involved (Lipson et al 2007).

None of these patents claim a bactericidal or bacteriostatic action.

The merit of this invention is to provide a new avenue for making use of cranberry marc to easily obtain an extract comprising proanthocyanidins, especially type A, with antibacterial, antiviral, antioxidant and anti-adhesive properties, all at the same time, presented in different galenic or other forms, used as a preventive or curative treatment of diseases of the teeth and oropharynx.

In one embodiment, the production method uses techniques that are not used in the prior state of the art; it allows a cranberry marc extract to be obtained in powder form, the composition of which was not previously known and that is richer in active substances than those obtained usually.

### Obtaining Proanthocyanidins From Cranberry Marc

According to the invention, the proposed technique to extract total proanthocyanidins from cranberries comprises several steps. The first step consists of obtaining the cranberry marc by pressing the Vaccinium macrocarpon and/or Vaccinium oxycoccus berries, which allows a large proportion of the carbohydrates and organic acids present in the juice to be removed; thus, the preparation obtained, with a pH varying from 3.5 to 4.5, is better accepted.

The cranberry marc used has an average solids content of 30%, where this rate may vary between 25 and 35% depending on the pressure applied during the pressing operation. It comes in the form of split berries and the seeds can be preserved since they contain proanthocyanidins. For ease of transport and storage, this marc has been frozen.

In a second step, proanthocyanidins are extracted using an aqueous or a hydroalcoholic solution. The passage of the extracts in an adsorption resin column can better isolate proanthocyanidins, thus increasing the extraction yield.

1^{st} case: the extraction is performed using an aqueous solution at ambient temperature or in water previously heated to between 50 and 70° C. The marc is first ground to increase the solid-liquid exchange surface and thus the extraction yield, which increases from 10 to 20% with the equipment used and a particle size below 2 mm.

The extraction itself can be performed on concentrations of marc of about 5 to 20% compared to that of water, the ideal proportion being 10%.

When working with heat, in water at 60° C., the marc, introduced at a rate of 10% in a tank fitted with a stirring mechanism is subjected to a constant stirring speed of between 200 and 500 rpm, depending on the type of blade used. The extraction can continue from 2 to 8 hours, but experience has shown that a duration of 5 hours is the most appropriate so as not to increase the proportion of water soluble fibers that harm the total concentration of active substances in the extract. At the end of the five hours, the suspension eventually undergoes coarse filtration and solid-liquid separation on a horizontal flow sedimentation tank running at a speed ranging from 2,500 and 6,500 rpm; with the equipment used, the best results were obtained at 3,500 rpm. This protocol is valid whatever the temperature of the water. The clarification of the suspension then continues with centrifugation carried out at a speed of between 4,000 and 8,000 rpm. In the next phase, the liquid extract thus obtained is subjected to desiccation, either by vacuum drying in an oven at a temperature below 60° C. so as not to degrade the active substances, or by lyophilization or even by atomization or even nebulization in order to obtain a powdery solid phase. Higher rates of active substances in the final product are obtained when a passage over adsorption resin follows centrifugation.

According to the invention and whatever the method used, the extraction yield is greater than 10% in relation to the amount of initial dry raw material.

The percentage of dry matter in the final product is above 90%, usually between 94 and 97%. The proanthocyanidins dosage can be achieved by the Vanillin-acid method or by the Bate Smith method, both well known.

After grinding, the fine and homogeneous powder obtained is pink to purple in color.

2^{nd} case: in this case the proanthocyanidins are extracted at ambient temperature, using a hydroalcoholic solution whose percentage of ethanol is variable, from 20 to 80%. Optimal conditions were obtained with 70% ethanol. The selected operating conditions are almost identical to the previous case: the cranberry marc is introduced at a rate of 10% into the medium. The stirring time of the suspension of 3 to 5 hours is also the most appropriate; this suspension is then subjected to decantation under the same conditions as above. At this stage, an improvement in the quality of the product can be obtained by centrifugation and then passage over adsorption resin. This allows higher proportions of active substances to be achieved; in this case, however, the alcohol must be completely removed before passing “the extract” over the adsorption resin. Similarly, drying is carried out in a vacuum oven at a temperature not exceeding 55° C. or by lyophilization. A variant of these methods that allow a powdery solid phase to be achieved also consists in subjecting the suspension to atomization.

The extraction yield is also about 10% dry powder from the initial dry matter.

The percentage of dry matter in the final product is above 90%, usually between 94 and 97%.

The dosage of proanthocyanidins is also performed by the Vanillin-acid or Bate-Smith method

After grinding, the fine and homogeneous powder is pink to purple in color.

### Improvement in the Proanthocyanidins Yield by Passage Over an Adsorption Resin

Principle: this operation, when properly controlled, allows the extraction yield to be increased. It consists of separating the anthocyanins and proanthocyanidins from the other undesirable substances contained in the liquid extract obtained after depletion of the cranberry marc with water at 60° C. or with the hydroalcoholic solution. After undergoing clarification by coarse filtration, decantation and then by centrifugation, the polyphenols contained in the liquid extract after removal of the alcohol, if it contains any, will adsorb on the resin and the remainder will be eliminated. Then simply make an elution to recover the active substances.

The absorption columns used are made of type 316L stainless steel, filled with FPX 66 grafted silicon dioxide type adsorption resin, or any other resin with equivalent properties. The resins are activated in a 96% ethanol bath for 24 hours minimum. They are then rinsed with water until the total elution of the alcohol.

Separation of the active substances: the column prepared for this purpose contains a volume BV (bed volume) of adsorbent resin. A volume BV of the liquid cranberry extract is loaded, against the current, from the bottom of the column at a speed of 2BV/hour. The water in which the resin soaks is removed from the top. Similarly, the non-fixed fraction, directly eluted is recovered. Once the volume BV of the liquid extract has been loaded, a water rinse is performed, this time from the top of the column at a rate of 2 BV/hour. It is designed to elute the unadsorbed compounds onto resins, particularly the non-phenolic substances and sugars. This rinse is continued until the solids content of the eluate is below 0.5% which corresponds approximately to a 3 BV volume of water.

Recovery of polyphenols adsorbed on the resins is then performed with 75% ethanol introduced at the top of the column at a rate of 2 BV/hour. The recovery of polyphenolic compounds is continued until the purple color completely disappears from the eluate.

The volume generally required to elute all the polyphenols adsorbed on the resin is approximately 2 BV. At this point, a dosage of the polyphenols is desirable to ensure that all the polyphenols, which are at least of the order of 20% of the amount of initial dry matter, have been eluted.

The recovered alcoholic eluate is then concentrated under partial vacuum at a temperature below 55° C.; simultaneously, regularly adding water into this hydroalcoholic medium prevents any risk of deterioration of the polyphenols. At the final stage, the proanthocyanidins are in a 100% aqueous medium. As previously mentioned, the product then undergoes desiccation in an oven under a partial vacuum or preferably lyophilization. It can also undergo atomization.

At this stage, the fine powder, purple to black in color, contains at least 20% of total proanthocyanidins. Depending on the extraction method and assay method used, the percentage of total proanthocyanidins (PACs) obtained in the final dry extract, i.e. the average purity rates are as follow:
Vanillin-acid method:
   1 to 10% PACs on dry matter for aqueous extraction
   10 to 20% PACs on dry matter for alcoholic extraction
   Over 20% PACs on dry matter for extraction after passage over resin.
Bate Smith method:
   5 to 20% PACs on dry matter for aqueous extraction
   10 to 30% PACs on dry matter for alcoholic extraction
   Over 40% and even over 50% PACs on dry matter for extraction after passage over resin.

### Applications

The extract of cranberry marc, mainly because it is rich in proanthocyanidins, particularly of type A, soluble at various pH (and therefore in the saliva) and having antibacterial, antiviral, antioxidant and anti-adhesive properties, can be used at different concentrations and galenic or various other forms, preventively or curatively, to fight against diseases of the teeth or of the oropharynx in humans and animals.

The final product can be presented as finely ground, granulated or encapsulated, notably in the form of tablets, orodispersible tablets, capsules, tablets made from gum arabic. Given its compatibility with most excipients used in pharmaceuticals, this type of preparation may notably, without problem, be flavored by fruit flavoring or essential oils, sweetened with aspartame, acesulfame, stevia rebaudiana, colored with authorized food coloring. Similarly, as it is also compatible with gelling agents such as pectins, gums (xanthan, gum arabic, gellan), dextrins, emulsifiers, polysaccharides, glucomannans, polyols such as sorbitol, xylitol, maltilol, lactilol, it can be included, for example, in chewing gum tablets containing polyols and glycerin, incorporated in toothpaste in the form of pastes or gels containing carboxyvinyl resins, mouthwashes by dissolving in glycerinated water and generally, in any solid, liquid or paste form, dispensed orally and enabling its release and dissolution in the mouth and oropharynx.

The effectiveness of these proanthocyanidins on oral bacteria has been proven by antibacterial tests and adhesion tests performed with three types of cranberry extracts whose concentrations are respectively 2 mg/ml, 5 mg/ml and 10 mg/ml, named Exocyan cran 1, Exocyan cran 2 and Exocyan cran 10 for this experiment; each of these extracts was prepared from proanthocyanidins solids titrated to 1%, 2% and 10%, which means for example, that series Exocyan cran 1, prepared from solids at 1% yielded three types of extracts at the respective concentrations of 2 mg/ml, 5 mg/ml and 10 mg/ml.

### Antibacterial Tests:

The effectiveness of cranberry extracts can be evaluated in vitro. The products are considered to have a bacteriostatic character when the number of germs decreases by at least 1/10 (a log vs. control), or are considered bactericidal when there is complete inhibition of the bacterial culture.

If the extracts are considered active in vitro compared to a control medium reproducing the saliva medium, it is expected that they will have the same activity in vivo; saliva, providing the dispersion of proanthocyanidins in the oral cavity, throat and digestive system, enables the active substances to be in contact with mucosal cells and to inhibit the formation of the biofilm.

Results: it was found that the three extracts at a concentration of 10 mg/ml have bacteriostatic activity on Streptococcus mutans, which suggests they have a bactericidal activity at a higher concentration (dose-dependent effect).

Only the extract Exocyan cran 10, at the concentration of 10 mg/ml, has a bacteriostatic activity on Porphyromonas gingivalis.

Fusobacterium nucleatum was the bacterium most sensitive to the three extracts at a concentration of 10 mg/ml; Exocyan cran 1 and Exocyan cran 10 have a bactericidal activity while Exocyan cran 2 shows a bacteriostatic activity.

The growth of Lactobacillus rhamnosus is not affected by any of the extracts, and this irrespective of their concentration.

Verification of the Anti-Adhesive Effect:

Bacteria have the ability to adhere to one another and form a biofilm in which the microorganisms that cause caries, gingivitis and periodontal disease proliferate.

It is considered that there is a positive result when a total or partial inhibition of the biofilm occurs (scores from 0 to 3), the lowest inhibition being obtained with the highest score.

It was observed that the three extracts at concentrations of 5 mg/ml have an effect on the biofilm of Streptococcus mutans; the same is true for Exocyan cran 1 and 10 at a concentration of 10 mg/ml (score=1); however, Exocyan cran 1 showed a lower inhibitory activity (score=3) than the other extracts.

1-9. (canceled)

10. Method for obtaining an extract of cranberry wherein an extract of cranberry marc, comprising a high percentage of proanthocyanidins, of type A in particular, variable depending on the methods of extraction and assay used and on whether it has been subjected to purification or not—that may be higher than 20% with the vanillin-acid method and higher than 40% or even 50% with the Bate Smith method—, having antibacterial, antiviral and anti-adhesive properties, is obtained from the cranberry berries—genus Vaccinium macrocarpon and/or Vaccinium oxycoccus—, by pressing the berry, then extracting the proanthocyanidins contained in the marc thus obtained using an aqueous solution either hot or at ambient temperature or a hydroalcoholic solution at ambient temperature, this operation being followed by known solid-liquid separation techniques, then by a passage over grafted silica type adsorption resin to separate the anthocyanins and proanthocyanidins from undesirable substances and to recover these same proanthocyanidins from the eluate in dry powder form by vacuum drying or lyophilization or atomization or nebulization.

11. Method for obtaining an extract of cranberry according to claim 10, wherein the extract of cranberry marc, comprising a high percentage of proanthocyanidins, of type A in particular, variable depending on the methods of extraction and assay used and on whether it has been subjected to purification or not—that may be higher than 20% with the vanillin-acid method and higher than 40% or even 50% with the Bate Smith method—, is obtained by aqueous extraction of proanthocyanidins from the ground cranberry marc, then mixed in a ratio of 5 to 20% with water at ambient temperature or heated to a temperature of between 50 and 70° C., the whole being stirred at a constant speed of between 200 and 500 rpm continuously for 2 to 8 hours; the water-soluble substances containing the proanthocyanidins being then separated from the insoluble material by coarse filtration, passage through a horizontal flow sedimentation tank with a rotation speed of between 2,500 and 6,500 rpm, centrifugation, then being isolated by passage over grafted silica type adsorption resin, recovered and dried by known appropriate desiccation techniques—vacuum drying or lyophilization or atomization or nebulization—to obtain a powdery solid phase.

12. Method for obtaining an extract of cranberry according to claim 10, wherein the extract of cranberry marc, comprising a high percentage of proanthocyanidins of type A in particular, variable depending on the methods of extraction and assay used and on whether it has been subjected to purification or not—that may be higher than 20% with the vanillin-acid method and higher than 40% or even 50% with the Bate Smith method—, is obtained by hydroalcoholic extraction of proanthocyanidins from the ground cranberry marc mixed at a rate of 5 to 20% into the aqueous solution containing 20 to 80% ethanol, the whole being stirred at a constant speed of between 200 and 500 rpm at ambient temperature, continuously for 2 to 8 hours, the soluble substances containing the proanthocyanidins being then separated from the insoluble material by coarse filtration, passage through a horizontal flow sedimentation tank with a rotation speed of between 2,500 and 6,500 rpm, centrifugation, then being isolated by a passage over grafted silica type adsorption resin, recovered and dried by known appropriate desiccation techniques—vacuum drying, lyophilization, atomization or nebulization—to obtain a powdery solid phase.

13. Method for obtaining an extract of cranberry according to claim 11, wherein the cranberry extract, obtained by aqueous extraction of the proanthocyanidins from the ground cranberry marc, is mixed in an ideal ratio of 10% with water at ambient temperature or heated to an ideal temperature of 60° C., the whole being stirred at a constant speed of between 200 and 500 rpm continuously for an ideal duration of five hours, the water soluble substances containing the proanthocyanidins being then separated from the insoluble material by coarse filtration, passage through a horizontal flow sedimentation tank with an ideal rotation speed of 3,500 rpm, centrifugation, then being isolated by passage over grafted silica type adsorption resin, recovered and dried by known appropriate desiccation techniques—vacuum drying or lyophilization or atomization or nebulization—to obtain a powdery solid phase.

14. Method for obtaining an extract of cranberry according to claim 12, wherein the cranberry extract, obtained by hydroalcoholic extraction of the proanthocyanidins from the ground cranberry marc then mixed in the ideal ratio of 10% into the aqueous solution ideally containing 70% ethanol, the whole being stirred at a constant speed of between 200 and 500 rpm at ambient temperature for an ideal duration of 5 hours, the soluble substances containing the proanthocyanidins being then separated from the insoluble material by coarse filtration, passage through a horizontal flow sedimentation tank with an ideal rotation speed of 3,500 rpm, centrifugation, then being isolated by a passage over grafted silicon dioxide type adsorption resin, recovered and dried by known appropriate desiccation techniques—vacuum drying, lyophilization, atomization or nebulization—to obtain a powdery solid phase.

15. Method for obtaining an extract of cranberry according to claim 10, wherein the increase in the percentage of active substances in the final product, and therefore in the yield of proanthocyanidins extracted from the cranberry marc, is obtained by passage of the extract over grafted silica adsorption resin, after centrifugation.

16. Method according to claim 10, wherein, after passage over grafted silica type adsorption resin, the yield of the extraction obtained by using an aqueous solution or using a hydroalcoholic solution is 10% greater relative to the quantity of initial dry matter, and the percentage of dry matter in the final product is above 90%.

17. Cranberry extract, obtained from cranberry marc according to claim 10, comprising a high percentage of proanthocyanidins of type A in particular, variable according to the methods of extraction and assay used and on whether it has been subjected to purification or not, having antibacterial, antiviral and anti-adhesive properties, that can be used at various concentrations, associated with pharmaceutical excipients and in various galenic or other forms such as tablets, sticks of chewing gum, toothpastes, mouthwashes, preventively or curatively, for the treatment of diseases of the teeth or of the oropharynx in humans and animals.

18. Preparation containing a cranberry extract wherein the extract of cranberry marc obtained according to claim 10, containing a high percentage of proanthocyanidins of type A in particular, variable depending on the methods of extraction and assay used and on whether it has been subjected to purification or not, having antibacterial, antiviral and anti-adhesive properties, is used at different concentrations and in various galenic or other forms in any solid, liquid or paste form dispensed orally and allowing the release and dissolution in the mouth and the oropharynx, for the preventive or curative treatment of diseases of the teeth or of the oropharynx in humans and animals.

19. Method according to claim 16, wherein the percentage of dry matter in the final product is between 94 and 97%.

20. Method according to claim 11, wherein the increase in the percentage of active substances in the final product, and therefore in the yield of proanthocyanidins extracted from the cranberry marc, is obtained by passage of the extract over grafted silica adsorption resin, after centrifugation.

21. Method according to claim 12, wherein the increase in the percentage of active substances in the final product, and therefore in the yield of proanthocyanidins extracted from the cranberry marc, is obtained by passage of the extract over grafted silica adsorption resin, after centrifugation.
